(19) United States
(12) Patent Application Publication
(10) Pub. No.: US 2019/0227023 A1
(43) Pub. Date: Jul. 25, 2019

(54) Biomarkers and label-free nucleic acid biomarker detection in cancer and other diseases

(71) Applicant: Detroit R&D Inc, Detroit, MI (US)

(72) Inventor: Hyesook Kim, Bloomfield Hills, MI (US)

(73) Assignee: Detroit R&D, Inc., Detroity, MI (US)

(21) Appl. No.: 15/850,222

(22) Filed: Dec. 21, 2017

(51) Int. Cl. G01N 27/327; C12Q 1/686; G01N 27/403; A01K 67/027

(52) U.S. Cl. CPC G01N 27/3278; C12Q 1/686; G01N 27/403; A01K 2267/0331; G01N 2800/7028; G01N 2800/60; G01N 2800/56; A01K 67/0276

(57) ABSTRACT

The present invention provides a method of obtaining miRNA biomarkers by data-search of up-regulated microRNAs (miRNAs) in cancer and screening the miRNA biomarkers to identify cancer kind- and stage-specific miRNA biomarker(s) using cancer cells, tissues and biological fluids. This invention includes obtaining lung cancer-specific miRNA biomarkers including early (Stage I) lung cancer miRNA biomarkers and identification of anti-cancer miRNA (complementary miRNA, DNA and their derivatives) in order to ameliorate diseases. This invention relates to novel utility of nanowell electrodes to detect target molecules in biological samples. The invention teaches methods to increase sensitivity of nanowell electrodes to detect target molecules including miRNA, RNA and DNA in biological samples to diagnose cancer and other diseases and monitoring prognosis and response to therapy. This invention includes methods to study loss of function of miR486, miR29c or miR122 in lung cancer cells, animal models and patients and find therapeutic anti-miRNA(s).

### GOVERNMENT SUPPORT

Research in this application was supported, in part, by contracts from National Cancer Institute (NCI Contract HHSN261201500040C).

### BACKGROUND OF THE INVENTION

### 1. Technical Field

The present invention provides method of obtaining miRNA biomarkers by data search of up-regulated cancer microRNAs (miRNAs) and screening the miRNA biomarkers to identify cancer kind- and stage-specific miRNA biomarker(s) using cancer cells and media and tissues and biological fluids. This invention includes obtaining lung cancer kind- and stage-specific miRNA biomarkers including early (Stage I) lung cancer miRNA biomarkers. This invention includes identification of anti-cancer miRNA (complementary miRNA, DNA and their derivatives) in order to use it to ameliorate or cure diseases. This invention also relates to methods to increase sensitivity of nanowell electrodes to detect miRNA, RNA and DNA in cell extracts and biological fluids using label-free technology and use of the microwell and nanowell technology for diagnosis of cancer and other diseases and monitoring prognosis and response to therapy of the diseases.

### 2. Background Art

Lung cancer is a leading cause of cancer death for both men and women in the United States, which causes more deaths than the combined deaths of the next most common cancers (colon, breast and pancreas). Cigarette smoking is a No. 1 risk factor of lung cancer. Approximately 160,000 Americans were predicted to die from lung cancer in 2014 (˜27% of all cancer deaths) (1). The majority (>75%) of lung cancer patients are diagnosed at an advanced or metastatic stage and their 5-year survival rate is <5%. However, lung cancer patients diagnosed at an early stage, Stage I, have about 70% 5-year survival rate (2), suggesting that early detection is an important strategy to lower lung cancer death rate.

The National Lung Screening Trial (NLST) at 33 US centers from August 2002 through April 2004 with ˜50,000 current and former smokers (high risk group) demonstrated that 3 yearly-screenings using low-dose computed tomography (LDCT) of the chest resulted in reduced lung cancer mortality by ˜20% compared with screening by chest x-ray (2,3). A few drawbacks of the LDCT were high screening cost, need for a skilled technician and high false positive rate (˜1 out of 4), which led to additional CT scanning, needle biopsies or even surgery of the cancer-free person.

Approximately 30% of human protein-encoding genes are regulated by mature miRNA. So far >900 miRNAs have been discovered (4). The mature miRNA is of 20-24 nucleotides (nt) in length (5) and is produced from a ˜70 nt long precursor RNA (pre-RNA) originating from a longer 300-5,000 nt primary RNA (pri-RNA) (6).

An miRNA down-regulates its target gene expression by degrading the target mRNA or inhibiting protein translation after binding within the 3′-untranslated sequence of the target mRNA, resulting in cell differentiation, proliferation or apoptosis. The function of each miRNA has been found to be conserved among many species (6) and differential expressions of miRNA were found under physiological and in pathological human conditions, including cancer. Aberrant miRNA expression has been reported in various cancerous tissues, serum/plasma, urine and other body fluids of cancer patients, including cancers of the lung, breast, prostate and gastro-intestine (4).

There are miRNAs up-regulated in both tissues and plasma in most types of cancers, i.e., miR-21, which down-regulate tumor suppressor genes such as PTEN (4,7,8). Recently, a few serum/plasma miRNAs have been identified as non-invasive biomarker candidates unique to each type of cancer.

Early detection of lung cancer is challenging yet important since diagnosis at earlier stages improves patient survival rates. A number of studies sought to identify substantial changes in cell-free miRNA levels during early stage lung cancer. Heegaard et al. (9) compared plasma samples of 220 early stage NSCLC patients and 220 healthy subjects and identified 7 down-regulated miRNAs and only one up-regulated miRNA (miR-29c) in lung cancer patients. In addition, using qRT-PCR, miR-30d and miR-21 were shown to increase in serum of lung cancer patients compared to healthy subjects (10,11). Altogether, detection of these miRNAs in plasma/serum could be an important strategy for early lung cancer diagnosis.

Recent studies at our laboratory using lung cancer miRNA biomarkers identified through data-searches (Table 1) (Example 2) revealed that increased levels of miR486 (Sequence ID 10), miR29c (Sequence ID 3) and miR122 (Sequence ID 5) and decreased levels of miR203 (Sequence ID 7) and miR205 (Sequence ID 8) are serum biomarkers and a signature of early (Stage I) lung cancer (FIG. 3) (Example 4).

Quantitative reverse-transcription PCR (qRT-PCR) of miRNAs using Taqman® (11-13) or a microarray of fluorescently labeled miRNAs (14,15) has been widely used for quantification of serum miRNAs. The qRT-PCR/Taqman® method requires an expensive instrument and fluorescence-dependent technology is not favorable due to intrinsic problems of high back-ground and low reproducibility.

Thus, much effort has been concentrated toward the development of a label-free electrochemical technology. The electrochemical biosensor detects an electrical change due to hybridization of a target molecule with a capture molecule, i.e., a protein, a DNA or an RNA biomarker in serum, which binds to an antibody or a complementary DNA probe coated on a nanogold surface in a nanowell (16).

Recently, a label-free 500 nm (diameter) nanowell electrode was fabricated and sensitivity of the electrode was determined by screening synthetic H5N1 DNA at concentrations from 1 pM to 1 μM using the electrode coated with complementary DNA (cDNA) of H5N1 influenza A viral DNA. The limit of detection (LOD) of the H5N1 target DNA by the 500 nm electrode was 1 pM (16).

Our company fabricated 90 nm and 200 nm nanowells, which increased the sensitivity for detection of miRNA (<1 fM) more than 1000-fold compared to the sensitivity of the 500 nm nanowell electrode (FIGS. 1 and 2, FIGS. 4 and 5) (Example 1).

Our laboratory found that levels of miR486 and miR29c (Sequence ID 3) increased in A549 human lung cancer cells but not in ACHN human kidney cancer cells using the qRT-PCR/Taqman® (Applied Biosystems) method (Table 3 and FIG. 2, Panel A) and, using the high sensitivity miRNA nanowell technology, we verified that miR29c (Sequence 3) levels in the lung cancer cells were 12.5-fold higher compared with the levels in kidney cancer using the 90 nm electrochemical analysis (FIG. 2, Panel B) (Example 3).

For the first time, 90 nm and 200 nm electrodes were used to detect increased levels of miR486 (Sequence ID 10) and miR29c (Sequence ID 3) in serum samples obtained from lung cancer patients and these miRNAs were found to be lung cancer biomarkers (FIG. 4-6) (Examples 5 and 6). These miRNAs which increased in adenocarcinoma Stages I-Ill and/or squamous cell carcinoma (SCC) non-small cell lung cancer (NSCLC) are especially useful as early lung cancer biomarkers, which increase in adenocarcinoma Stage I patients compared to healthy subjects.

### SUMMARY OF INVENTION

The present invention provides a method of obtaining miRNA biomarkers by data search of up-regulated cancer miRNAs and screening the miRNA biomarkers to identify cancer kind- and stage-specific miRNA biomarker(s) using cancer cells and media and tissues and biological fluids. This invention includes obtaining lung cancer kind- and stage-specific miRNA biomarkers including early (Stage I) lung cancer miRNA biomarkers. This invention includes identification of anti-cancer miRNAs (complementary miRNA, DNA and their derivatives) in order to ameliorate diseases. This invention also relates to methods to increase sensitivity of nanowell electrodes to detect miRNA, RNA and DNA using label-free technology and use of the electrodes to diagnose cancer and other diseases and monitoring the prognosis and response to therapy of the diseases.

### DESCRIPTION OF THE DRAWINGS

Other advantages of the present invention will be readily appreciated as the same becomes better understood by reference to the following detailed description when considered in connection with the accompanying drawings wherein:

FIG. 1 is a graph showing representative Nyquist impedance plots using various concentrations of miR486 standards (1 fM, 100 fM and 10 pM) (Sigma) obtained by electrochemical analysis using a 90 nm nanowell array electrode (Detroit R&D) coated with biotinylated cDNAs. The impedance level increased following binding of miR486 (Sequence. 10) to the cDNA probe (Sequence ID 21) in a PBS solution containing ferricyanide/ferrocynide (5 mM each)/potassium chloride (100 mM) using a potentiometer (Ivium Technology). The limit of detection (LOD) of the miRNAs using nanowell electrodes was <1 fM;

FIGS. 2A and 2B show results from a lung (A549) and kidney (ACHN) cell culture study. Panel A, levels of miR29c (Sequence ID 3) in lung and kidney cancer cells and media detected by qRT-PCR/Taqman® analysis (5 ng total RNA/sample) and Panel B(i) is a graph showing representative Nyquist impedance plots of with (1 fM) and without (cDNA) miR29c miRNA standard (Sigma). The biotinylated miR29c cDNA (Sequence 14) was obtained from Integrated DNA Technologies. The limit of detection (LOD) of the miRNAs using nanowell electrodes was <1 fM. Panel B(ii), a graph showing an increase in impedance levels for miR29c in the A549 lung cancer cell media as compared to levels using kidney cell media using 8-channel 90 nm nanowell electrodes (Detroit R&D). The impedance level increased after miR29c was bound to the cDNA probe as determined in a PBS solution containing ferricyanide/ferrocynide (5 mM each)/potassium chloride (100 mM) using a potentiometer (Ivium Technology);

FIGS. 3A, 3B, 3C and 3D show the up- and down-regulation of miRNAs in Stages I, II and III adenocarcinoma and/or squamous cell carcinoma (SCC) non-small cell lung cancer (NSCLC, ˜80% of lung cancer cases). Levels of miR486 (Sequence ID 10) (Panel A), miR203 (Sequence ID 7) (Panel B) and miR205 (Sequence ID 8) (Panel C) in serum samples obtained from 11 healthy subjects, 21 Stage I, 8 Stage II and 11 Stage III and 5 squamous cell carcinoma (SCC) lung cancer patients using qRT-PCR/Taqman® analysis (6 ng total RNA/sample). Each target miRNA level is normalized by miR16 (internal control) level. Then the negative of each value was taken and normalized by a mean −ΔCt calculated from all patients and healthy subjects. *p<0.05 from normal samples. miR486 was up-regulated in Stages I, II and III adenocarcinoma and SCC lung cancer (Panel A). Panel D, detection of miR29c (Sequence ID 3) and miR122 (Sequence ID 5) in serum samples using qRT-PCR/Taqman® analysis;

FIGS. 4A and 4B show levels of miR486 (Sequence ID 10) in serum samples obtained from healthy subjects (pooled serum sample, control) and a lung cancer patient (Innovative Research). Panel A, PCR results obtained by qRT-PCR/Taqman® (6 ng total RNA/sample) are shown as a fold-increase from control samples. Panel B(i), a graph of representative Nyquist impedance plots of 1 fM miR486 (Sequence 10) standard (Sigma) obtained by electrochemical analysis using a 90 nm nanowell array electrode (Detroit R&D) coated with biotinylated miR486 cDNAs (Sequence ID 21). Panel B(ii), representative Nyquist impedance plots of control and lung cancer serum total RNA samples (50 ng/μl) (1 μl/channel) incubated 1 hr with miR486 biotinylated cDNA-streptavidin (Sequence ID 21) on a SAM/gold 90 nm nanowell electrode. SAM (self-assembly monolayer) was produced on nanowells by incubating 10 mM of 11-mercaptoundecanoic acid (MUA) (Sigma). Panel B(iii), Quantitation of impedance results .from 8 individual channels and results of statistical T-test using the Salstat2 software carried out to verify differences between the control and lung cancer serum samples;

FIGS. 5A and 5B show levels of miR29c (Sequence 3) in serum samples obtained from healthy subjects (pooled serum sample, control) and a lung cancer patient (Innovative Research). Panel A, PCR results obtained by qRT-PCR/Taqman® are shown as a fold-increase from control samples. Panel B(i), a graph showing representative Nyquist impedance plots of 1 fM miR29c (Sequence ID 3) standard (Sigma) obtained by electrochemical analysis using a 90 nm nanowell array electrode (Detroit R&D) coated with biotinylated miR29c cDNAs (Sequence 14). Panel B(ii), representative Nyquist impedance plots of control and lung cancer serum total RNA samples (50 ng/μl) (1 μl/channel) incubated 1 hr with miR29c biotinylated cDNA-streptavidin on a SAM/gold 90 nm nanowell electrode. Panel B(iii), Quantitation of impedance results from 8 individual channels and results of statistical T-test using the Salstat2 software carried out to verify differences between the control and lung cancer serum samples; and

FIGS. 6A and 6B show electrochemical analyses of miR486 (Sequence ID 10) levels with a pooled control serum sample and a lung cancer serum sample (Innovative Research) without isolation of total RNA. Panel A, representative Nyquist impedance graphs with addition of control and lung cancer serum samples (1 μl each). Panel B, Quantitation of impedance levels from normal and lung cancer serum samples and a graph of mean value±SD from data obtained by 4 channels each of control and lung cancer serum samples. The serum sample (100 μl) was sonicated 5 times for 3 seconds to burst exosomes. The electrode was incubated for 1 hr with biotinylated miR486 cDNAs (Sequence ID 21)-streptavidin on a SAM/gold nanowell electrode. Redox conversion of the electrode was determined using a buffer containing ferricyanide/ferrocynide (5 mM each) and potassium chloride (100 mM) in PBS using an Ivium potentiometer (Ivium Technology). CV analysis settings were −0.5 V E start, 0.5 V Vertex1, −0.5 V Vertex2, 10 E step, 3 N scans and 50 mV/s scan rate. Control and lung cancer samples were compared by T-test.

### DETAILED DESCRIPTION OF THE INVENTION

The present invention includes a method of identifying single or multiple miRNA biomarker(s) to diagnose various stages and kinds of lung cancer followed by experimental screening of a group of 9 lung cancer miRNA biomarkers (Table 1) (Sequences 1-8 and 10) found by data-searches of up-regulated miRNA expression in lung cancer cells, tissues or biological fluids of various phases of lung cancer (Example 2).

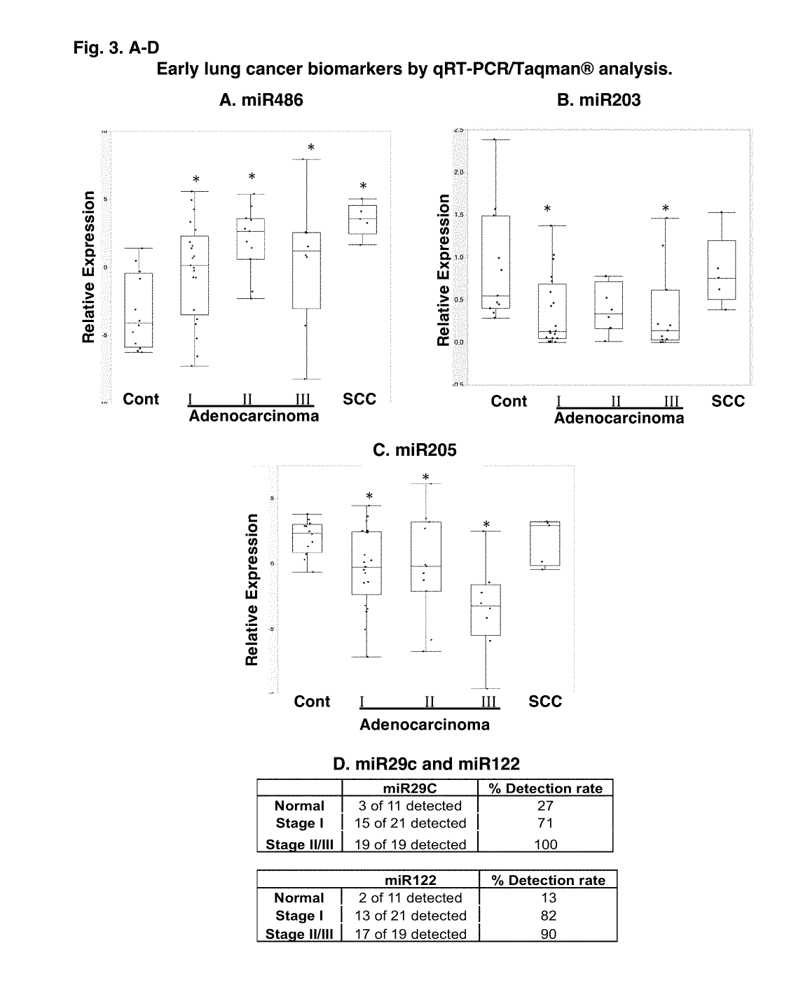

Use of an innovative method of an extensive data-search (Table 1) followed by experimental selection of the target biomarkers resulted in finding early lung cancer biomarkers of up-regulated miR486 (Sequence ID 10) and/or miR29c (Sequence ID 3) and miR122 (Sequence 5) using serum samples obtained from 11 healthy subjects, 21 Stage I, 8 Stage II and 11 Stage III adenocarcinoma and 5 squamous cell carcinoma (SCC) lung cancer patients (FIG. 3). Addition of down-regulated miR203 (Sequence ID 7) to the up-regulated biomarkers and normalization of levels of the biomarkers using the level of miR16 (internal control) (Sequence ID 11) further improved accuracy of the diagnosis (Examples 2 and 3).

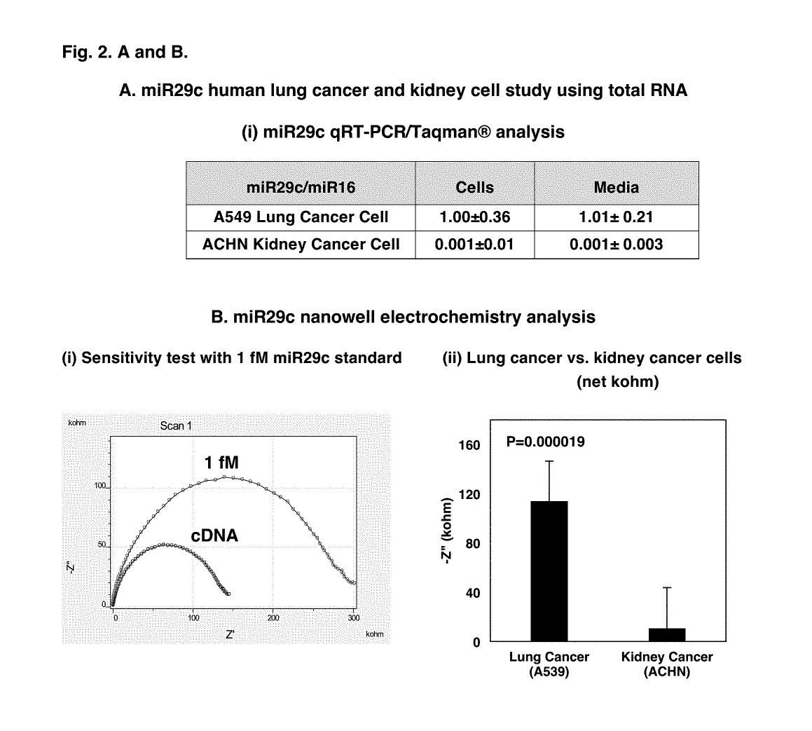

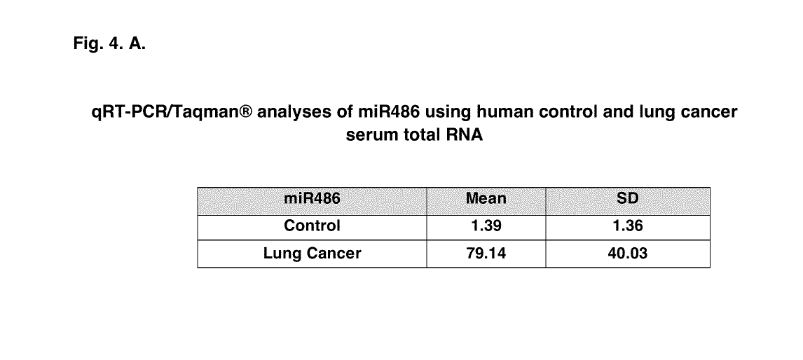

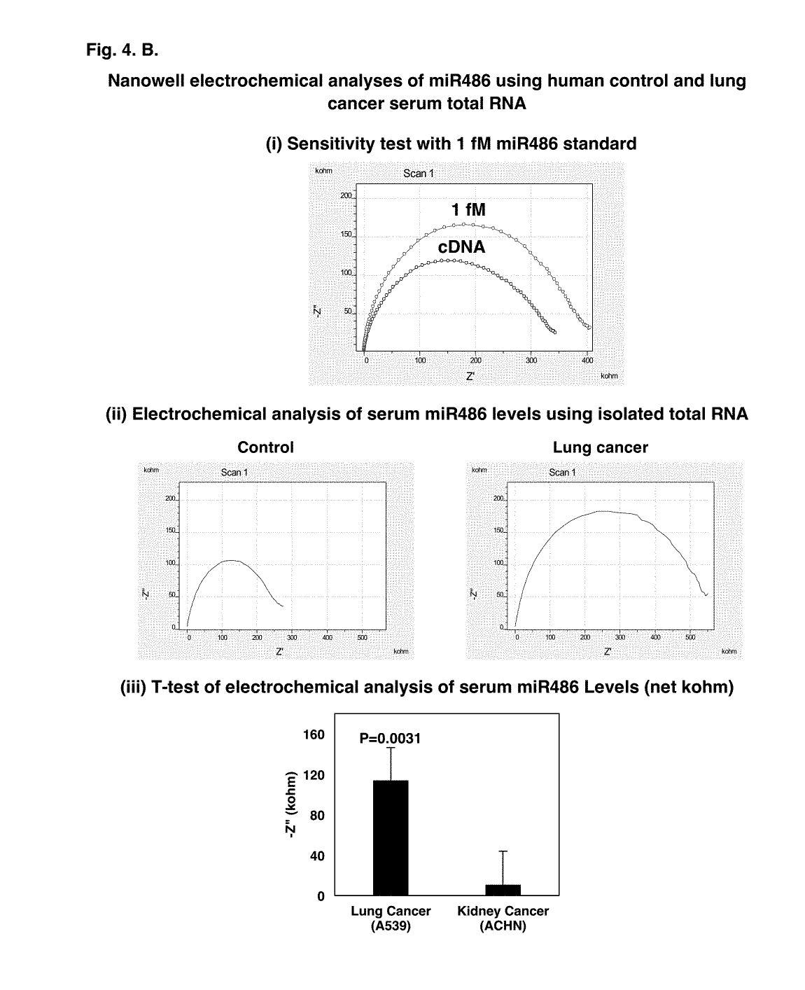

Moreover, increased levels of miR486 (Sequence ID 10) and miR29c (Sequence ID 3) normalized to levels of miR16 (Sequence ID 11, internal control without change) in lung cancer were verified by (a) a lung and kidney cancer study (Table 3 and FIG. 2) and (b) a second human serum study carried out at our laboratory using human control (pooled from 52 Afro Americans+26 Caucasians, 47 males+31 females) and lung cancer serum samples obtained from Innovative Research (Novi, Mich.) (FIG. 4-7).

This method can be used to obtain miRNA biomarkers for other cancers or diseases.

By the data-search of miRNAs up-regulated in various kinds and phases of lung cancer, e.g., early, prognosis, metastasis and proliferation, a group of 9 lung cancer miRNA biomarkers, miR21 (Sequence ID 1), miR25 (Sequence ID 2), miR29c (Sequence ID 3), miR30d (Sequence ID 4), miR122 (Sequence ID 5), miR195 (Sequence ID 6), miR205 (Sequence ID 8), miR411 (Sequence ID 9) and miR486 (Sequence ID 10), were obtained (Table 1). Among them, 8 biomarkers (Table 2, Sequences 1-6, 8 and 10) except for miR411 (Table 2, Sequence ID 9) were identified as lung cancer biomarkers by at least two different laboratories (Table 1) (Example 2).

Eight miRNAs containing 6 biomarkers selected from the group of the up-regulated lung cancer biomarkers identified by at least two different laboratories (Table 2, Sequences 1-6 and 8-10), miR203, a down-regulated lung cancer biomarker (17) (Table 2, Sequence ID 7), and miR16, an internal control (Sequence ID 11) (Table 2) were used to screen for highly expressed miRNA biomarker(s) in A549 lung cancer cells as compared with ACHN kidney cancer cells using qRT-PCR/Taqman® analysis (Table 3). Ratios of miR486 (Sequence ID 10)/miR16 (Sequence ID 11) and miR29c (Sequence ID 3)/miR16 (Sequence ID 11) among the up-regulated miRNAs were much higher in A549 lung cancer cell lysates and media compared to ratios of the miRNAs in ACHN kidney cell lysates and media (Table 3, FIG. 2, Panel A) (Example 3).

qRT-PCR of miRNAs using Taqman® (11-13) is widely used but it requires an expensive instrument and significant cost. Thus a facile label-free electrochemical technology was developed and carried out to detect DNA using an electrode containing 500 nm (diameter) nanowells (16). The LOD of the assay using H5N1 DNA standards was 1 pM which is not suitable for miRNA biomarker screening. The LOD of the assay has to be at least 1 fM to detect miRNAs in serum/plasma samples. Electrodes containing 50 nm, 90 nm and 200 nm (diameter) nanowells, which are smaller than 500 nm (diameter) (16,18), were developed by Detroit R&D (Example 1) and use of the electrodes containing nanowells smaller than 500 nm increased sensitivity of the device (LOD of <1 fM).

This is a surprising finding that sensitivity of the label-free nanowell technology for detection of target molecules in fluids can be increased by using an electrode containing nanowells with a diameter smaller than 500 nm but same or larger than 50 nm.

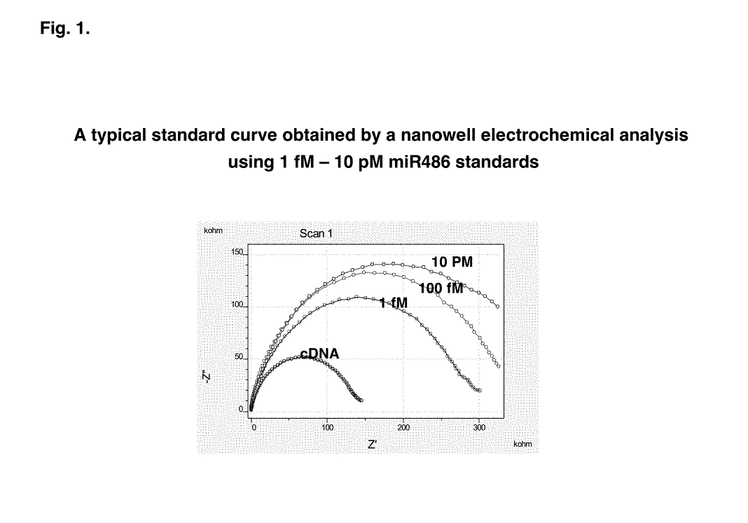

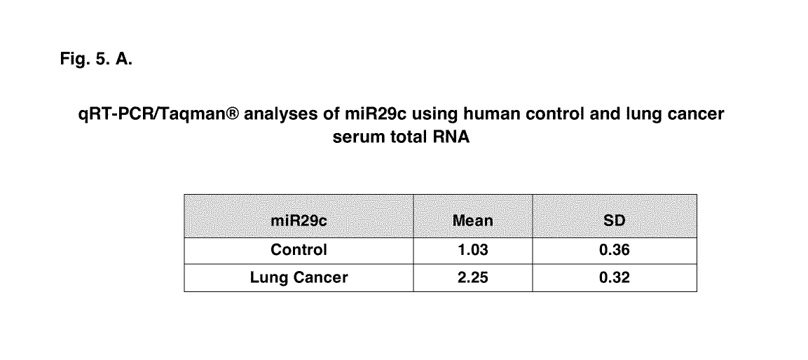

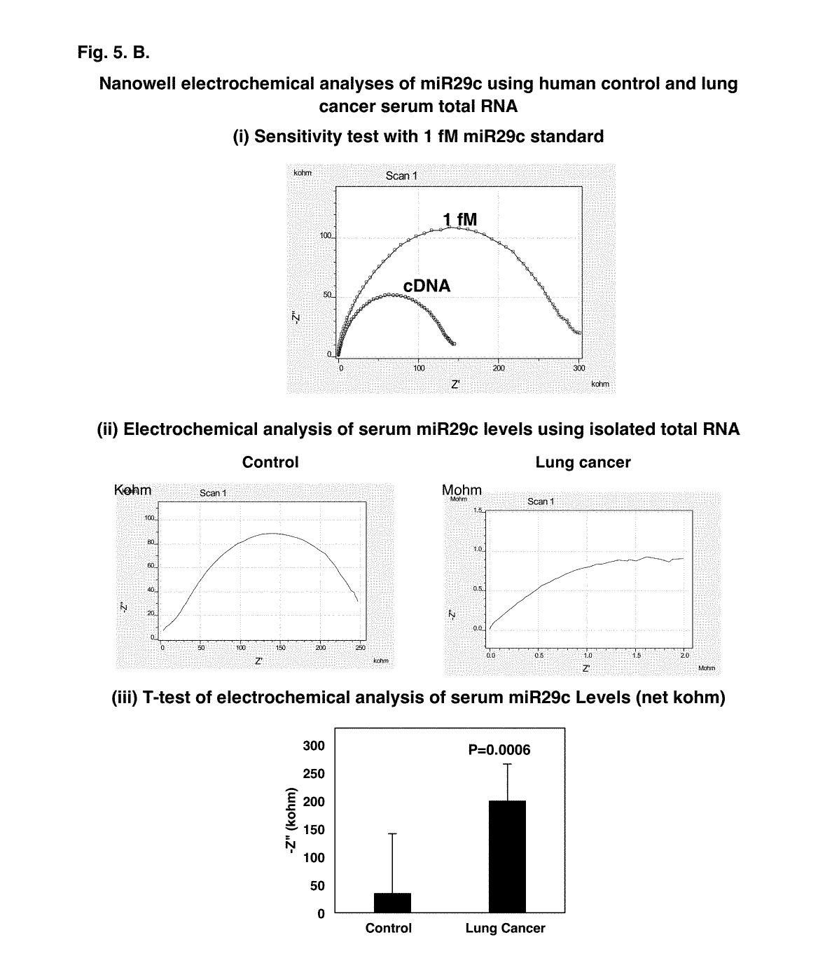

For example, the 500 nm (diameter) nanowell which has nano-gold surface area of 196.3 nm² (250 nm×250 nm×3.14) is 30.7-fold larger than the 90 nm nanowell (diameter), which has 6.4 nm² (45 nm×45 nm×3.14). The smaller the nanowell gold surface, the higher the specificity of the nanowell technology because it can prevent non-specific binding of molecules bigger than the target miRNA to the biotinylated cDNA probe captured by the streptavidin on the nano-gold surface.

Whereas the LOD of the 90 nm nanowell electrode was expected to be 32.5 fM according to the calculation shown above, the LOD values of miR486 (Sequence ID 10) (FIGS. 1 and 4, Panel B) and miR29c (Sequence ID 3) (FIGS. 2 and 5, Panel B) early lung cancer biomarkers and miR16 internal control (Sequence ID 11) at our laboratory were <1 fM.

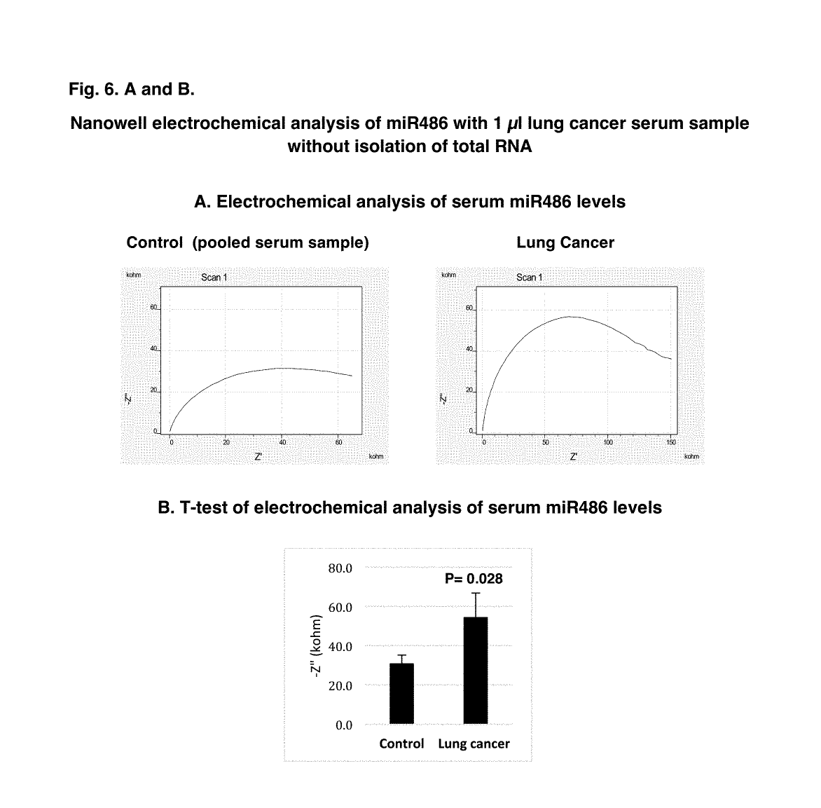

Due to the unique feature of the high sensitivity nanowell technology, the mi486 level, which was higher than the pooled control blood sample, was successfully determined using 1 μl lung cancer blood sample without isolation of total RNA (FIG. 6).

Whereas the number of nanowells per unit of the 500 nm nanowell electrode are larger compared to the number of nanowells per unit of the 90 nm or 200 nm nanowell electrode, the LOD of the 500 nm nanowell electrode, 1 pM, was much higher than the LOD of 90 nm or 200 nm nanowell electrode, 1 fM (FIG. 1, FIG. 2, FIG. 4 and FIG. 5).

Levels of miR29c (Sequence ID 3) in A549 lung cancer and ACHN kidney cancer cell media were measured using all 8 channels of a 90 nm nanowell array electrode (Detroit R&D) coated with biotinylated miR29c cDNA (Sequence ID 14). Impedance levels of miR29c (−Z″ kohm) in A549 lung cancer cell media were 12.5-fold higher in impedance levels compared with the ACHN kidney cancer cell media (FIG. 2, Panel B).

The 9 lung cancer miRNA biomarkers (Sequence IDs 1-8 and 10) found by data-search of up-regulated miRNAs at our laboratory were selected as biomarkers specific for lung cancer (Table 1). However, the experiment carried out at our laboratory revealed that only miR486 and miR29c were lung cancer-specific biomarkers in both cells and media when their levels were compared to levels in ACHN kidney cell lysates and media (Table 3 and FIG. 2) (Example 3). This correlated well with the results obtained with the human serum studies using human serum samples obtained from two different sources carried out at our laboratory (FIG. 3-7).

Thus this invention reveals that lung cancer cells including A549 cells can be used to study loss of function of the miRNA biomarkers up-regulated in lung cancer cells compared with negative control cells including other cancer cells or primary cells

Due to the correlation of the up-regulation of miR486 or miR29c in lung cancer cells with the up-regulation in human lung cancer serum samples, characterization of the effect of the anti-miRNA (complementary miRNA, DNA and their derivatives) treatment to find therapeutic anti-miRNAs using the cancer cells became feasible.

The miR411 (Sequence ID 8) was deleted from the 9 miRNA biomarkers shown in Table 1 because only one laboratory has reported it as a lung cancer biomarker. Moreover, the miR411 (Sequence ID 8) previously found in lung cancer tissues (Table 1) was not detected in either cell lysates or media of human lung (A549) and kidney (ACHN) cancer cells (Table 3). The result suggests that miR411 is most likely not secreted to blood in lung cancer (Example 2).

The majority (>75%) of lung cancer patients are not diagnosed until an advanced or metastatic stage and their 5-year survival rate is <5% (2). Early (Stage I) lung cancer miRNA biomarker screening will increase 5-year survival rate of lung cancer patients because lung cancer patients diagnosed at early stage, Stage I, have about 70% 5-year survival rate (1).

A human lung cancer serum study was carried out using samples obtained from serum samples from 11 healthy subjects, 21 Stage I, 8 Stage II and 11 Stage III adenocarcinoma and 5 squamous cell carcinoma (SCC) lung cancer patients (in total, 56 human serum samples) using qRT-PCR/Taqman® analysis (FIG. 3) (Example 4).

Total RNA fractions of the serum samples were separated by the TRIzol® method. Using the 56 total RNA fractions from the serum samples, levels of, in total, 8 miRNAs, miR21 (Sequence ID 1), miR25 (Sequence ID 2), miR29c (Sequence ID 3), miR122 (Sequence ID 5), miR205 (Sequence ID 8), miR41.1 (Sequence ID 9) and miR486 (Sequence ID 10) up-regulated miRNAs, miR203 (Sequence ID 7) down-regulated miRNA and, an internal control miRNA, miR16 (internal control) (Table 1), were determined by qRT-PCR/Taqman® analysis (in duplicate, in total, 1,026 analyses). The level of each miRNA biomarker candidate in a serum sample was normalized by the level of miR16 and/or total signal level.

Levels of miR486, miR122 and miR29c were found up-regulated and levels of miR203 and miR205 were found down-regulated in adenocarcinoma in Stage I as well as in Stages II or III (FIG. 3). The miRNA biomarkers can be used for early (Stage I) lung cancer diagnosis.

The miR486/miR16 values of adenocarcinoma Stage I (p=0.0145), Stage II (p=0.0004) and Stage III (p=0.0174) differed significantly from healthy subjects (p=0.0145). The miR486/miR16 value of SCC patients differed significantly from healthy subjects (p=0.0003) (FIG. 4).

Electrochemical analyses of a serum sample obtained from a lung cancer patient (Innovative Research) using a 90 nm nanowell electrode revealed that miR486 (Sequence 10) (FIG. 4, Panel B) and miR29c (Sequence 3) (FIG. 5, Panel B) levels were ˜6-fold (p=0.0031) and 27.5-fold (p=0.0006) higher, respectively, compared to the pooled human control sera (Innovative Research, pooled from 52 Afro Americans+26 Caucasians, 47 males+31 females). miR486 and miR29c levels in the human serum sample were also higher than the control serum by qRT-PCR/Taqman® analysis (Example 5).

miR29c expression levels in both cell lysates and cell culture media were higher in the A549 lung cancer cells compared to the expression in ACHN kidney cells by qRT-PCR/Taqman® (Table 3), which were confirmed by qRT-PCR/Taqman® and nanowell assays of human lung cancer plasma samples (FIG. 5).

The miR16 level in the serum sample obtained from a lung cancer patient wasn't higher than the level in the pooled control serum in electrochemical or qRT-PCR/Taqman® analyses.

Our results suggest that up-regulated miR486, miR29c and/or miR122 with and without down-regulated miR203 or miR205 in lung cancer serum samples are biomarkers for early (Stage I) lung cancer diagnosis. The miR122 miRNA was expressed in 82% of Stage I adenocarcinoma serum samples and in only 13% of control samples and miR29c miRNA was expressed in 71% of Stage I adenocarcinoma serum samples and 27% in control samples (FIG. 3, Panel D).

Our results showed that single or multiple measurement of up-regulated miR486, miR29c or miR122 are biomarker(s) for early lung cancer. Dividing the levels of up-regulated miRNA biomarker(s) by the levels of down-regulated miR203 or miR205 of a patient which is higher than the control can be used as a biomarker. In addition, the concentration of miR486, miR29c or miR203 can be normalized by the concentration of an miR16 internal control which had a minimal change in lung cancer.

It was surprising that the serum/plasma miR486 level previously found as a biomarker of lung cancer prognosis by 2 laboratories (Table 1) and the plasma and cell miR122 level previously found as a biomarker for metastasis and prognosis (Table 1) were identified as early lung cancer biomarkers (FIG. 3, Panels A and D, respectively).

The nanowell electrodes used for the miRNA study contain thousands of nanometer (diameter)-sized wells, which prevented any particles larger than the diameter of the nanowell from binding to the nanowell. The nanowell technology increases sensitivity by increasing the signal/noise ratio.

Because of this unique feature of nanowells, miRNAs in biological fluids including urine and blood samples may be used without isolation of total RNA prior to the measurement (Example 6).

Recent electrochemical analysis of miR486 levels was carried out at our laboratory with 1 μl serum sample obtained from a healthy subject and a lung cancer patient (Innovative Research) without isolation of total RNA. The result revealed that (a) the impedance signal obtained from the 1 μl/test serum sample as it is (˜30 kohm) (FIG. 7) was weaker than the signal obtained with 50 ng total RNA/μl (1 μl/test) (130 kohm) (FIG. 4, Panel B) for the human pooled control serum sample and (b) the miR486 level of the serum sample obtained from the lung cancer patient was higher compared with the level of the pooled control serum sample (FIG. 6).

The increase of miR486 in cancer also agreed with the result obtained by qRT-PCR/Taqman® using 6 ng total RNA/sample and nanowell electrochemical analyses of the human serum samples using 50 ng total RNA/μl (1 μl/test) (FIG. 4).

Isolation of serum/plasma total RNA is required for miRNA detection by qRT-PCR/Taqman® analysis. Our results demonstrated utility of the nanowell array technology to detect miRNA biomarkers in serum samples without isolation of total RNA.

Anti-miRNAs (antisense oligonucleotides) can be used as anti-cancer molecules (19). Effects of the anti-miRNAs on cancer can be studied using cancer cells or animal disease models and used to treat patients. Loss of function of miR486, miR29c or miR122 in lung cancer cells and lung cancer animal models and patients can be studied to characterize the effect of the anti-miRNA (complementary miRNA, DNA and their derivatives) treatment and find therapeutic lung cancer anti-miRNA(s).

Most of the techniques used for screening of miRNA biomarkers and anti-miRNAs in the present disclosure are practiced in the art, and most practitioners are familiar with the standard resource materials, which describe specific conditions and procedures. The methods used with and the utility of the present invention can be shown by the following non-limiting examples and accompanying figures.

### EXAMPLES

Materials and Methods

Materials.

Total RNA was isolated from cell lysates and media and serum samples obtained from University of Michigan (Ann Arbor, Mich.) and Innovative Research (Novi, Mich.). qRT-PCR/Taqman® (Applied Biosystems) analyses were carried out with 5 ng total RNA/sample for the cell study and with 6 ng total RNA/sample for miRNA lung cancer biomarkers and normalized by the miR16 level (no change reported in lung cancer) or total signals. Novel label-free 8-channel nanowell electrodes were developed by Detroit R&D, Inc., producing the electrodes at the Lurie Nanofabrication Facility (University of Michigan). miRNAs for the nanowell array analysis were obtained from Sigma (MISSION RNAi product family). Biotinylated DNAs were synthesized at Integrated DNA Technologies (Coralville, Iowa) (Table 2).

Methods.

Electrochemical analysis: Electrochemical analyses of nanowell electrodes were carried out as previously described (16,18) with modifications. Electrical current of the nanowell electrodes was confirmed by standard cyclic voltammetry (CV) measurement of the bare electrode by dipping the electrode in a PBS solution containing ferro/ferricyanide (5 mM each), 100 mM KCl using an 8-channel Ivium potentio-n-stat (Ivium Technology). Current/potential analyses were carried out with each channel of an 8-channel electrode. The settings for the CV using a bare electrode were −0.5 V E start, 0.5 V Vertex1, −0.5 V Vertex2, 10 E step, 3 N scans and 50 mV/s Scan rate. Similar levels of current (pA) for each channel of the 8-channel nanowell electrode was detected demonstrating production of a high quality nanowell electrode.

After CV analyses, a self-assembly monolayer (SAM) was produced on the 700 μm² nano-gold surface of each channel by incubating 10 mM of 11-mercaptoundecanoic acid (MUA) (Sigma) for 1 hr at room temperature. After washing the electrode with ethanol and phosphate-buffered saline (PBS), 50 mM EDC and 50 mM NHS in sodium acetate buffer (pH 5.5) was added to form active ester functional groups and kept for 15 minutes. After washing electrodes with PBS, streptavidin (Sigma) diluted in PBS (10 Ng/ml) was added and incubated for an hour. The streptavidin-coated nanowell array electrodes were bound with 5 μM biotinylated cDNA probe complementary to each miRNA (Integrated DNA Technologies, Coralville, Iowa) (Table 2) for 1 hr and rinsed 3 times with PBS. Reference, counter and working electrodes were immersed in the ferricyanide solution. Impedance measurements (−Z″ vs. Z′) were obtained after binding of biotinylated cDNA probe to a streptavidin-coated nanowell electrode. To quantify the hybridization between the cDNA and miRNA, charge transfer resistances (electrochemical impedances) were measured using Ivium-n-Stat potentiometer (Ivium Technology). The impedance resulting from the miRNA bound to biotinylated cDNA captured by streptavidin to the SAM/nanogold surface was determined by redox conversion using ferricyanide in PBS. The settings were −0.5 V E start, 0.5 V Vertex1, −0.5 V Vertex2, 10 E step, 3 N scans and 50 mV/s Scan rate.

Cell culture: Cells were grown in 100 mm Petri dishes at 37° C. under a 5% CO₂ atmosphere. A549 lung cancer cells (ATCC) were grown in a formulated medium (Cat. #30-2004) purchased from ATCC and human epithelial cancer kidney cells (ACHN) (ATCC) in EMEM with 10% serum and 0.1% antibiotics. Cells were collected with trypsin and centrifuged for 5 min at 1,000×g. The pellet washed with PBS twice and 3 ml of cell media was collected for RNA isolation.

RNA isolation using TRIzol®/chloroform (Invitrogen): Total RNAs from the harvested cells, media and human serum samples were isolated using a TRIzol® methodology according to the manufacturer's protocol (Invitrogen, Life Technologies). The RNA concentration was determined.

Quantification using the qRT-PCR/Taqman® (Applied Biosystems) assays: qRT-PCR/Taqman® analysis was performed according to instruction provided by the manufacturer.

### Example 1

Production of 50 nm, 90 nm and 200 nm (Diameter) Nanowell Array Electrodes. Novel label-free 50 nm, 90 nm and 200 nm (diameter) 8-channel nanowell electrodes were developed by Detroit R&D, Inc. and produced at the Lurie Nanofabrication Facility (University of Michigan). The 50 nm, 90 nm and 200 nm nanowell electrodes were fabricated as previously described for the 500 nm 2-channel electrode (16,18) but with modifications of e-beam patterning of 50 nm, 90 nm and 200 nm nanowells in a space of 700 μm² nano-gold and increasing the electrode from 2 channels to an 8-channel multiplex electrode.

### Example 2

Data-Search of Lung Cancer miRNA Biomarkers.

Identification of 9 miRNA biomarkers by data-search was carried out using search engines including Google and Pubmed for various key words pertinent to lung cancer miRNAa up-regulated in lung cancer cells, tissues or biological fluids in various kinds and phases of lung cancer, e.g., early, prognosis, metastasis and proliferation. Nine miRNA biomarkers were found as shown in Table 1. A group of 9 lung cancer miRNA biomarkers, miR21 (Sequence ID 1), miR25 (Sequence ID 2), miR29c (Sequence ID 3), miR30d (Sequence ID 4), miR122 (Sequence ID 5), miR195 (Sequence ID 6), miR205 (Sequence ID 8), miR411 (Sequence ID 9) and miR486 (Sequence ID 10), were obtained. Among them, 8 biomarkers (Sequence IDs 1-6, 8 and 10) (Table 2) (Sequence ID 9) were identified as lung cancer biomarkers by at least two different laboratories. The exception was miR411 (Table 1).

### Example 3

Experimentally Screening miRNA Biomarkers Identified Through Data-Searches (Table 1) Using Human Lung (A549) Cells with Negative Control Kidney (ACHN) Cancer Cells by qRT-PCR/Taqman® and Nanowell Analyses.

Expression levels of 8 miRNAs, the 6 miRNA lung cancer biomarkers, miR21 (Sequence ID 1), miR25 (Sequence ID 2), miR29c (Sequence ID 3), miR122 (Sequence ID 5), miR411 (Sequence ID 9) and miR486 (Sequence ID 10), with down-regulated miR203 (Sequence ID 7) and an internal control, miR-16 (Sequence ID 11) (Table 2) in lung and kidney cancer cells and media were measured by qRT-PCR/Taqman® analysis to experimentally identify a group of miRNA biomarkers for the human cell study.

qRT-PCR/Taqman® analysis was performed with lung and kidney cancer cell lines (A549 and ACHN, respectively) using 5 ng of total RNA. The assay was performed in triplicate for each sample. Results are expressed as meant standard deviation calculated by the comparative Ct method (ΔΔCt method) in which the target miRNAs were normalized by the endogenous control (miR16). The differences were then converted to fold-differences. Ratios of miR486/miR16 and miR29c/miR16 increased more than 10-fold in lung cancer cell lysates and media compared to ratios of the miRNAs in kidney cell lysates and media (Table 3). Whereas the miR203 level, which has been known to decrease in lung cancer, increased in the human lung cancer cells and media compared to the level in kidney cell and media (Table 3), the miR203 level decreased in human lung cancer serum samples compared with the level in control serum samples (FIG. 3). The results demonstrated that miR-486 and miR29c, which increased in both human lung cancer cell (Table 3) and serum (FIG. 3) studies, are lung cancer-specific miRNA biomarkers.

The miR411 (Sequence ID 8) was deleted from the 9 miRNA biomarkers shown in Table 1 because only one laboratory has reported it as a lung cancer biomarker and the miR411 (Sequence ID 8) was not detected in cell media of A549 human lung cancer cells (Table 3) suggesting that the miR411 is most likely not secreted to blood in lung cancer.

Levels of miR29c in the A549 and ACHN cell media were measured using all 8 channels of a 90 nm electrode (FIG. 2, Panel B). Impedance levels (−Z″ kohm) of miR29c bound to biotinylated cDNA/streptavidin on the SAM/gold surface of 8 channels were measured using an Ivium-n-Stat potentiometer. Impedance levels of miR29c in the A549 lung cancer cell media were 12.5-fold higher compared with the ACHN kidney cancer cell media. Levels of miR486 in the A549 cell lysates were also measured using 8 channels of a 90 nm electrode. The results demonstrated that miRNAs expression in A549 lung cancer cells can be studied using the 90 nm nanowell technology.

### Example 4

A Human Lung Cancer Serum Study Using Serum Samples from Various Stages of Adenocarcinoma and Squamous Cell Carcinoma (SCC) Lung Cancer Patients Using qRT-PCR/Taqman® Analysis

Early (Stage I) lung cancer miRNA biomarker screening will increase 5-year survival rate of lung cancer patients (˜70% 5-year survival rate) (1).

A human lung cancer serum study was carried out using serum samples from 11 healthy subjects, 21 Stage I, 8 Stage II and 11 Stage III adenocarcinoma and 5 squamous cell carcinoma (SCC) lung cancer patients (in total, 56 human serum samples) using qRT-PCR/Taqman® analysis (FIG. 3). Total RNA fractions of the serum samples were separated by the TRIzol® method. Using the 56 total RNA fractions from the serum samples, levels of 8 miRNAs, miR21 (Sequence ID 1), miR25 (Sequence ID 2), miR29c (Sequence ID 3), miR122 (Sequence ID 5), miR 203 (Sequence ID 7), miR205 (Sequence ID 8), miR411 (Sequence ID 9) and miR486 (Sequence ID 10) and miR16 (internal control) (Sequence ID 11) (Tables 1 and 2) were determined by qRT-PCR/Taqman®analysis (in duplicate, in total, 1,026 analyses). The level of each miRNA biomarker in a serum sample was normalized by the level of miR16 and/or total signal level.

Levels of miR486, miR29c and miR122 were found to be up-regulated (FIG. 3), which can be used for early (Stage I) lung cancer diagnosis as single or a combination of 2 or 3 miR biomarkers. Inclusion of levels of down-regulated miR203 and miR205 in the serum sample of a patient with the up-regulated miRNA biomarker(s) will increase specificity and sensitivity of early lung cancer diagnosis.

The miR486/miR16 values of adenocarcinoma Stage I (p=0.0145), Stage II (p=0.0004) and Stage III (p=0.0174) patients differed significantly from healthy subjects (p=0.0145) and the miR486/miR16 value of SCC patients differed significantly from healthy subjects (p=0.0003) (FIG. 3).

The miR21 level, significantly decreased only in adenocarcinoma Stage I patients, can be used to improve specificity of early lung cancer diagnosis.

### Example 5

A Human Lung Cancer Serum Study of miR486 (Sequence ID 10) and miR29c (Sequence ID 3), Early Lung Cancer Biomarkers by Lung Cancer Cell (Table 3 and FIG. 2) and Adenocarcinoma Stages I, II and III (FIG. 3) Serum Studies with Serum Samples from a Pooled Control and a Lung Cancer Patient Using Both qRT-PCR/Taqman® and Nanowell Electrochemical Analyses

qRT-PCR/Taqman® and nanowell electrochemical analyses were carried out for miR486 and miR29c (FIG. 4 and FIG. 5, respectively) which have been identified at our laboratory as lung cancer-specific miRNA biomarkers by the human lung and kidney cancer cell study (Table 3 and FIG. 2) and human adenocarcinoma Stages I, II and III and SCC lung cancer serum study (FIG. 3).

Both qRT-PCR/Taqman® and nanowell analyses were carried out for miR486 (FIG. 4) and miR29c (FIG. 5) using total RNA samples isolated from serum samples obtained from pooled healthy subjects (pooled from 52 Afro Americans and 26 Caucasians, 47 males and 31 females) and a lung cancer patient (Innovative Research).

qRT-PCR/Taqman® analyses in triplicate/sample were carried out with the total RNA (6 ng/sample) and a mean value of ΔΔCt of miR486/miR16 (FIG. 4, Panel A) or miR29c/miR16 (FIG. 5, Panel A) was calculated (miR16, an internal control). Both miR486 and miR29c levels increased in the lung cancer serum sample which strongly suggested that these two miRNAs are lung cancer biomarker(s) as single or as a combination of the two biomarkers.

For nanowell assay, sensitivity of the electrochemical analysis of the 90 nm nanowell electrode (Detroit R&D) was verified using 1 fM miR486 and miR29c standards (Sigma) (FIG. 4. Panel B and FIG. 5, Panel B, respectively).

Total RNA samples (50 ng/μl, 1 μl/test) isolated from serum samples obtained from the pooled control and a lung cancer patient (Innovative Research) were incubated for 1 hr with biotinylated miR486 (Sequence ID 21) or miR29c (Sequence ID 14) cDNA (Integrated DNA Technologies)/streptavidin conjugated on an SAM/gold 90 nm nanowell electrode. Nyquist plots were generated from impedance measurements (FIG. 4, Panel B and FIG. 5, Panel B, respectively). Impedance results for each channel of the 8-channel 90 nm electrode were quantified and statistical T-test from Salstat2 software was used to verify differences between control and lung cancer samples (FIG. 4, Panel B and FIG. 5, Panel B, respectively).

miR486 and miR29c levels in serum samples obtained from human lung cancer patients were ˜6-fold (p<0.0031) and 27.5-fold (p=0.0006) higher, respectively, compared to the pooled human control sera (FIG. 4 and FIG. 5, respectively).

Using this method, we also carried out miR16 (internal control) nanowell assays using total RNA isolated from serum samples obtained from healthy subjects (control) and lung cancer patients and no significant difference in miR16 levels between control and lung cancer was detected.

### Example 6

Electrochemical Analysis Using a Nanowell Electrode to Detect miR486 (Sequence ID 10), Identified as an Early Lung Cancer Biomarker at Our Laboratory, Using 1 μl Serum Sample without Isolation of Total RNA: A Technology Suitable for a Finger Prick Assay

Electrochemical analysis using a nanowell electrode was carried out to detect miR486 (Sequence ID 10) levels using 1 μl serum sample obtained from pooled control (52 Afro Americans and 26 Caucasians, 47 males and 31 females) and a lung cancer patient (Innovative Research) without isolation of total RNA (FIG. 6). Impedance levels of the control and lung cancer serum samples were determined (FIG. 6, Panel A) and a quantitative graph was generated from data obtained using 4 channels each of control and lung cancer serum samples (FIG. 6, Panel B). The serum samples were sonicated 5 times for 3 seconds to burst exosomes prior to the impedance measurement. The electrode was incubated for 1 hr with biotinylated miR486 cDNAs (Sequence 21)-streptavidin on a SAM/gold nanowell electrode. Redox conversion of the electrode was determined using an Ivium potentiometer. Control and lung cancer results were compared by T-test.

The result revealed a lung cancer-specific increase in impedance, suggesting that the nanowell structure can lower or eliminate background noise resulting from various particles in serum or blood.

Our results demonstrated a utility of the nanowell array technology to detect miRNA biomarkers in lung cancer serum samples.

TABLE 1
Nine lung cancer miRNA biomarker candidates and the miR-16 internal control
selected for lung cancer nanowell array analysis. Eight miRNA biomarkers,
Sequence IDs 1-7 and 9, were up-regulated in lung cancer cells and serum samples.
They can be used for development of lung cancer miRNA diagnostics.

| | | | Up | |
|---|---|---|---|---|
| | | | Regulation | |
| | | | or no | |
| miRNA | | Sample | change | Use as Biomarker |
| Lung | 1. miR-21 | Tumor Tissue^{7,10} Serum⁴ | Up^{3,4,7,10} | Early Detection^{3,11} |
| Cancer | | Plasma^{3,10,11} Cell lines¹⁰ | | Prognosis^{4,7,10} |
| Biomarker | 2. miR-25 | Plasma^{2,12} | Up^{2,12} | Metastasis¹² Prognosis² |
| Candidate | 3. miR-29c | Plasma¹³ Serum¹ | Up^{1,13} | Early Detection^{1,13} |
| | 4. miR-30d | Serum^{4,5} | Up^{4,5} | Early Detection^{4,5} |
| | 5. miR-122 | Plasma¹⁴ Cell⁸ | Up^{8,14} | Metastasis⁸ Prognosis¹⁴ |
| | 6. miR-195 | Plasma¹⁴ Cell⁹ Tumor Tissue⁹ | Up^{9,14} | Metastasis⁹ Proliferation⁹ |
| | | | | Prognosis¹⁴ |
| | 7. miR-205 | Plasma¹⁷ Tumor Tissue^{15,16}Cell¹⁷ | Up^{15,16,17} | Prognosis^{15,16,17} |
| | 8. miR-411 | Tumor Tissue⁷ | Up⁷ | Prognosis⁷, Metastasis⁷ |
| | 9. miR-486 | Plasma¹⁷ Serum⁴ | Up^{4,17} | Prognosis^{4,17} |
| Control | 10. miR-16 | Plasma³, Serum⁵ Tissue⁶ | No Change | Normalization Control^{3,5,6} |

¹Zhang J G, Wang J J, Zhao F. et al. Clin Chim Acta (2010) 411, 846-852;
²Chen X, Hu Z, Wang W, Ba Y, Ma L, Zhang C, Wang C, et al. Int J Cancer (2012) 130, 1620-1628;
³Wei J, Gao W, Zhu C J. et al. Chin J Cancer (2011) 30, 407-414;
⁴Hu Z, Chen X, Zhao Y. et al. J Clin Oncol (2010) 28, 1721-1726;
⁵Le H B, Zhu W Y, Chen D D. et al. Med Oncol. (2012) 5, 3190-7;
⁶Raponi M, Dossey L, Jatkoe T. et al. Cancer Res (2009) 69, 5776-5783;
⁷Nadal E, Zhong J, Lin J, Reddy R M. et al. Clin Cancer Res (2014) 20, 3107-3317;
⁸Ma L, Liu J, Shen J. et al. Cancer Biol Ther. (2010) 9, (7): 554-61;
⁹Yongchun Z, Linwei T, Xicai W. et al. Cancer Lett. (2014) May 28; 347(1): 65-74.
¹⁰Gao W, Lu X, Liu L. et al. Cancer Biol Ther (2012)13, 330-340;
¹¹Tang D, Shen Y, Wang M. et al. Eur J Cancer Prev (2013) 22, 540-548;
¹²Xu F X, Su Y L, Zhang H et al. Asian Pac J Cancer Prev (2014) 15, 1197-1203;
¹³Zhu W, He J, Chen D. et al. PLoS One (2014) 9, e87780;
¹⁴Zhang H, Su Y, Xu F. et al. PLoS One (2013) 8, e81408;
¹⁵Hamamoto J, Soejima K, Yoda S. et al. Mol Med Rep (2013) 8, 456-462;
¹⁶Zhang Y K, Zhu W Y, He J Y. et al. J Cancer Res Clin Oncol (2012) 138, 1641-1650 and
¹⁷Aushev V N, Zborovskaya I B, Laktionov K K. et al. PLoS One (2013) 8, e78649.

TABLE 2
Target miRNA and biotinylated complementary DNA sequences of 10
miRNAs for the lung cancer biomarker study (all miRNAs and DNAs are 22-mer except
for the 21-mer miR-195). The 10 miRNA sequences for the nanowell array analysis
were obtained through a program at Sigma (MISSION RNAi product family).

| miRNA | Target miRNA | Biotinylated Complementary DNA |
|---|---|---|
| 1. miR-21 | Sequence 1. 5′-UAGCUUAUCAGACUGAUGUUGA-3′ | Sequence 12. 5′-Bio-TCAACATCAGICTGATAAGCTA-3′ |
| 2. miR-25 | Sequence 2. 5′-CAUUGCACUUGUCUCGGUCUGA-3′ | Sequence 13. 5′-Bio-TCAGACCGAGACAAGTGCAATG-3′ |
| 3. miR-29c | Sequence 3. 5′-UGACCGAUUUCUCCUGGUGUUC-3′ | Sequence 14. 5′-Bio-GAACACCAGGAGAAATCGGTCA-3′ |
| 4. miR-30d | Sequence 4. 5′-UGUAAACAUCCCCGACUGGAAG-3′ | Sequence 15. 5′-Bio-CTTCCAGTCGGGGATGTTTACA-3′ |
| 5. miR-122 | Sequence 5. 5′-AACGCCAUUAUCACACUAAAUA-3′ | Sequence 16. 5′-Bio-TATTTAGTGTGATAATGGCGTT-3′ |
| 6. miR-195 | Sequence 6. 5′-UAGCAGCACAGAAAUAUUGGC-3′ | Sequence 17. 5′-Bio-GCCAATATTTCTGTGCTGCTA-3′ |
| 7. miR-203 | Sequence 7. 5′-GUGAAAUGUUUAGGACCACUAG-3′ | Sequence 18. 5′-Bio-CTAGTGGTCCTAAACATTTCAC-3′ |
| 8. miR-205 | Sequence 8. 5′-UCCUUCAUUCCACCGGAGUCUG-3′ | Sequence 19. 5′-Bio-CAGACTCCGGTGGAATGAAGGA-3′ |
| 9. miR-411 | Sequence 9. 5′-UAUGUAACACGGUCCACUAACC-3′ | Sequence 20. 5′-Bio-GGTTAGTGGACCGTGTTACATA-3′ |
| 10 miR-486 | Sequence 10. 5′-UCCUGUACUGAGCUGCCCCGAG-3′ | Sequence 21. 5′-Bio-CTCGGGGCAGCTCAGTACAGGA-3′ |
| 11. miR-16 | Sequence 11. 5′-UAGCAGCACGUAAAUAUUGGCG-3′ | Sequence 22. 5′-Bio-CGCCAATATTTACGTGCTGCTA-3′ |
| (Internal | | |
| control) | | |

TABLE 3
qRT-PCR/Taqman ® analysis of 7 miRNA lung cancer
biomarkers and an internal control, miR-16 expressed in
cell lysates and media obtained from human lung (A549) and
kidney (ACHN) cancer cells. Levels of miR-486/miR-16, miR-
29c/miR-16 and miR203/miR16 increased more than 10-fold
in lung cancer cell lysates and media compared to the ratios
of the miRNAs in kidney cell lysates and media (shaded).

| | Cells | Media |
|---|---|---|
| miR21/miR16 | | |
| A549 | 1.01 ± 0.20 | 1.08 ± 0.86 |
| ACHN | 1.87 ± 0.33 | 7.77 ± 0.88 |
| miR122/miR16 | | |
| A549 | Not-detected | 1.48 ± 1.46 |
| ACHN | Detected | 0.02 ± 0.01 |
| miR411/miR16 | | |
| A549 | Not-detected | Not-detected |
| ACHN | Not-detected | Not-detected |
| miR203/miR16 | | |
| A549 | 1.04 ± 0.43 | 1.01 ± 0.02 |
| ACHN | 0.01 ± 0.00 | 0.02 ± 0.01 |
| miR25/miR16 | | |
| A549 | 1.01 ± 0.08 | 1.08 ± 0.89 |
| ACHN | 0.25 ± 0.01 | 5.68 ± 0.43 |
| miR486/miR16 | | |
| A549 | 1.00 ± 0.02 | 1.01 ± 0.01 |
| ACHN | 0.08 ± 0.01 | 0.13 ± 0.03 |
| miR29c/miR16 | | |
| A549 | 1.00 ± 0.36 | 1.01 ± 0.21 |
| ACHN | 0.001 ± 0.01 | 0.001 ± 0.003 |

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### REFERENCES

1. http://www.lung.org/lung-disease/lung-cancer/resources/facts-figures/lung-cancer-fact-sheet.html.
2 National Lung Screening Trial Team, The national lung screening trial: Overview and study design. Radiology (2011) 258, 243-253.
3. National Lung Screening Trial Team, Results of initial low-dose computed tomographic screening for lung cancer. N Engl J Med (2013) 368, 1980-1991.
4. Gonzalez-Masia J A, Garcia-Olmo D, Garcia-Olmo D C. Circulating nucleic acids in plasma and serum (CNAPS): applications in oncology. Onco Targets Ther (2013) 6, 819-832 and references therein.
5. Chen X, Ba Y, Ma L, et al. Characterization of microRNAs in serum: a novel class of biomarkers for diagnosis of cancer and other diseases. Cell Res (2008)18, 997-1006.
6. Sassen S, Miska E A, Caldas C. MicroRNA: implications for cancer. Virchows Arch (2008) 452, 1-10.
7. Mo M H, Chen L, Fu Y, Wang W, Fu S W. Cell-free circulating miRNA biomarkers in cancer. J Cancer (2012) 3, 432-448.
8. Zhang J G, Wang J J, Zhao F, Liu Q, Jiang K, Yang G H: MicroRNA-21 (miR-21) represses tumor suppressor PTEN and promotes growth and invasion in non-small cell lung cancer (NSCLC). Clin Chim Acta (2010) 411, 846-852.
9. Heegaard N H, Schetter A J, Welsh J A, Yoneda M, Bowman E D, Harris C C. Circulating microRNA expression profiles in early stage non-small cell lung cancer. Int J Cancer (2012) 130, 1378-1386.
10. Wei J, Gao W, Zhu C J, Liu Y Q, Mei Z, Cheng T, Shu Y Q: Identification of plasma microRNA-21 as a biomarker for early detection and chemosensitivity of non-small cell lung cancer. Chin J Cancer (2011) 30, 407-414.
11. Le H B, Zhu W Y, Chen D D, He J Y, Huang Y Y, Liu X G, Zhang Y K. Evaluation of dynamic change of serum miR-21 and miR-24 in pre- and post-operative lung carcinoma patients. Med Oncol. (2012) 5, 3190-7.
12. Raponi M, Dossey L, Jatkoe T, Wu X, Chen G, Fan H, Beer D G. MicroRNA classifiers for predicting prognosis of squamous cell lung cancer. Cancer Res (2009) 69, 5776-5783.
13. Santos J M, Kowluru R A. Role of mitochondria biogenesis in the metabolic memory associated with the continued progression of diabetic retinopathy and its regulation by lipoic acid. Invest Ophthalmol Vis Sci. 2011 Nov. 11; 52(12):8791-8.
14. Leidinger P, Backes C, Blatt M, Keller A, Huwer H, Lepper P, Bals R, Meese E. The blood-borne miRNA signature of lung cancer patients is independent of histology but influenced by metastases. Mol Cancer (2014) 13, 202.
15. Wang K, Zhang S, Weber J, Baxter D, Galas D J. Export of microRNAs and microRNA-protective protein by mammalian cells. Nucleic Acids Res (2010) 38, 7248-7259.
16. Cha M S, Lee J K, Cho S H, Park J G, Lee H Y, Lim S H, Yoon Y R. Quantitative analysis of H5N1 DNA hybridization on nanowell array electrode. J Nanosci Nanotechnol (2013) 13, 5245-5249.
17. Wang N, Liang H, Zhou Y, Wang C, Zhang S, Pan Y, Wang Y, Yan X, Zhang J, Zhang C Y, Zen K, Li D, Chen X. miR-203 suppresses the proliferation and migration and promotes the apoptosis of lung cancer cells by targeting SRC. PLoS One. 2014 Aug. 20; 9(8):e105570.
18. Lee J, Cho S, Lee J, Ryu H, Park J, Lim S, Oh B, Lee C, Huang W, Busnaina A, Lee H. Wafer-scale nanowell array patterning based electrochemical impedimetric immunosensor. J Biotechnol. 2013 December; 168(4):584-8.
19. Stenvang J, Petri A, Lindow M, Obad S, Kauppinen S. Inhibition of microRNA function by antimiR oligonucleotides. Silence. 2012 Jan. 9; 3(1):1.

1. A method of increasing sensitivity of nanowell electrode analysis to detect molecules in fluids by using an electrode containing nanowells with the diameter of the nanowells smaller than 500 nm but same or larger than 50 nm including the steps of:
obtaining a wafer with metal surface, e.g., nano-gold;
spin-casting the wafer with electron beam resist;
producing nanowells smaller than 500 nm but same or larger than 50 nm on the wafer using an electron beam lithography system; and
measuring levels of the target molecule in a sample using potentiometer.

2. The method of claim 1, wherein the molecules in fluid is miRNA, RNA, DNA, protein or fatty acid.

3. The method of claim 2, wherein the fluid is a purified or as it is (not purified) biological fluid including cell extract, serum, plasma, blood or urine.

4. The method of claim 3, additionally including the steps of performing an electrochemical analysis of the sample.

5. The method of claim 3, wherein the diameter of nanowell is 50 nm-200 nm.

6. The method of claim 4, wherein the diameter of nanowell is 50 nm-200 nm.

7. A method to identify single or multiple miRNA biomarker(s) to diagnose various stages and kinds of lung cancer by experimentally screening a group of lung cancer miRNA biomarkers found by data search of up-regulated miRNAs expressed in lung cancer cells, tissues or biological fluids in various phases of lung cancer by the steps of:
identifying a group of lung cancer miRNA biomarkers which are up-regulated in various kinds and phases of lung cancer, e.g., early, prognosis, metastasis and proliferation, found by at least two different laboratories;
experimentally determining miRNA biomarker candidate levels in lung cancer cells and cell media and biological samples suitable for a targeted biomarker study with controls.
normalizing the levels by the miR16 level or total signal of control and experimental groups.
Identifying single or multiple up- and/or down-regulated miRNA biomarker(s) to diagnose the targeted lung cancer.

8. The method of claim 7, wherein a group of up-regulated miRNA biomarkers for various kinds and phases of lung cancer found by at least two different laboratories are miR21, miR25, miR29c, miR30d, miR122, miR195, miR205 and miR486 and a down-regulated miRNA biomarker is miR203 using various miRNA detection methods including qRT-PCR performed by TaqMan® miRNA assay, label-free electrochemical analysis or microarrays.

9. The method of claim 8, wherein miRNA biomarker(s) for lung cancer were experimentally determined using serum or plasma samples obtained from healthy subjects and adenocarcinoma patients at various stages including stages I and/or squamous cell carcinoma (SCC) non-small cell lung cancer (NSCLC) patients to find miRNA biomarker(s) increased or decreased in adenocarcinoma stage I as early lung cancer biomarker(s).

10. The method of claim 9, wherein miRNA biomarker(s) for early lung cancer are up-regulated miR486 and/or miR29c and miR122.

11. The method of claim 9, wherein miRNA biomarker(s) for lung cancer were experimentally determined using serum or plasma samples from lung cancer patients at adenocarcinoma stages I, II, III or IV to identify stage-specific miRNA biomarkers.

12. The method of claim 8, wherein miRNA biomarker(s) for lung cancer were experimentally determined using serum or plasma samples from various lung cancer patients to diagnose SCC or non-NSCLC patients.

13. The method of claim 8, wherein miRNA biomarker(s) for lung cancer were experimentally determined using serum or plasma samples from various lung cancer patients to diagnose poor prognosis, cancer metastasis or short or long life-expectancy.

14. A method to diagnose adenocarcinoma and/or squamous cell carcinoma (SCC) non-small cell lung cancer (NSCLC) including early lung cancer by the steps of:
measurements of miR486 levels of serum/plasma samples obtained from a person and control healthy subjects;
comparing the miR486 level of the person with the level of serum/plasma samples obtained from the control healthy subjects;
determining a person with an miR486 level higher than the control level as a lung cancer patient;

15. The method of claim 14, wherein the detection method is qRT-PCR/Taqman® analysis or nanowell electrochemistry.

16. The method of claim 15, wherein the miR486 level is normalized by miR16 levels.

17. A method to diagnose adenocarcinoma lung cancer including early lung cancer by the steps of:
measurements of miR486 and miR203 levels of serum/plasma samples obtained from a person and control healthy subjects;
comparing the miR486 and miR203 level of the person with the levels of serum/plasma samples obtained from the control healthy subjects;
determining a person with an miR486 level higher and an miR203 level lower than the control level as a adenocarcinoma lung cancer patient;

18. The method of claim 17, wherein the detection method is qRT-PCR/Taqman® analysis or nanowell electrochemistry.

19. The method of claim 18, wherein the miR486 and miR203 levels are normalized by miR16 levels.

20. A method to diagnose adenocarcinoma and/or squamous cell carcinoma (SCC) non-small cell lung cancer (NSCLC) including early lung cancer by the steps of:
measurements of miR486 and miR29c levels of serum/plasma samples obtained from a person and control healthy subjects;
comparing the miR486 and miR29c levels of the person with the levels of serum/plasma samples obtained from the control healthy subjects;
determining a person with miR486 and miR29c levels higher than the control level as a lung cancer patient;

21. The method of claim 20, wherein the detection method is qRT-PCR/Taqman® analysis or nanowell electrochemistry.

22. The method of claim 21, wherein the miR486 and miR29c levels are normalized by miR16 levels.

23. A method to diagnose adenocarcinoma lung cancer including early lung cancer by the steps of:
measurements of miR486, miR29c and miR203 levels of serum/plasma samples obtained from a person and control healthy subjects;
comparing the miR486, miR29c and miR203 levels of the person with the levels of serum/plasma samples obtained from the control healthy subjects;
determining a person with miR486 and miR29c level higher and an miR203 level lower than the control level as a adenocarcinoma lung cancer patient;

24. The method of claim 23, wherein the detection method is qRT-PCR/Taqman® analysis or nanowell electrochemistry.

25. The method of claim 24, wherein the miR486, miR29c and miR203 levels are normalized by miR16 levels.

26. A method to study loss of function of miR486 or miR29c in lung cancer cells and characterizing the effect of the anti-miRNA (complementary miRNA, DNA and their derivatives) treatment and finding therapeutic anti-miRNA, including the step of:
measurement of miR486 or miR29c levels of lung cancer cells and negative control cells including other cancer cells or primary cells;
treating the cells with anti-miRNA;
characterizing the effect of the anti-miRNA treatment, e.g., lung cancer cell-specific cell death;

27. The method of claim 26, wherein the loss of function study is carried out in animal lung cancer models including the step of:
measurements of miR486 or miR29c levels of serum/plasma samples obtained from animal lung cancer models;
treating animals with anti-miRNA;
characterizing the effect of the anti-miRNA treatment, e.g., suppression of cancer metastasis, reduction of angiogenesis and tumor burden;


SEQUENCE LISTING (22 sequences)

SEQ ID NO: 1 - RNA, 22 nt, Artificial Sequence
  synthetic
uagcuuaucagacugauguuga

SEQ ID NO: 2 - RNA, 22 nt, Artificial Sequence
  synthetic
cauugcacuugccucggucuga

SEQ ID NO: 3 - RNA, 22 nt, Artificial Sequence
  synthetic
ugaccgauuucuccugguguuc

SEQ ID NO: 4 - RNA, 22 nt, Artificial Sequence
  synthetic
uguaaacauccccgacuggaag

SEQ ID NO: 5 - RNA, 21 nt, Artificial Sequence
  synthetic
aacgccauuaucacacuaaau

SEQ ID NO: 6 - RNA, 21 nt, Artificial Sequence
  synthetic
uagcagcacagaaauauuggc

SEQ ID NO: 7 - RNA, 22 nt, Artificial Sequence
  synthetic
gugaaauguuuaggaccacuag

SEQ ID NO: 8 - RNA, 22 nt, Artificial Sequence
  synthetic
uccuucauuccaccggagucug

SEQ ID NO: 9 - RNA, 22 nt, Artificial Sequence
  synthetic
uauguaacacgguccacuaacc

SEQ ID NO: 10 - RNA, 22 nt, Artificial Sequence
  synthetic
uccuguacugagcugccccgag

SEQ ID NO: 11 - RNA, 22 nt, Artificial Sequence
  synthetic
uagcagcacguaaauauuggcg

SEQ ID NO: 12 - DNA, 22 bp, Artificial Sequence
  synthetic
tcaacatcagtctgataagcta

SEQ ID NO: 13 - DNA, 22 bp, Artificial Sequence
  synthetic
tcagaccgagacaagtgcaatg

SEQ ID NO: 14 - DNA, 22 bp, Artificial Sequence
  synthetic
gaacaccaggagaaatcggtca

SEQ ID NO: 15 - DNA, 22 bp, Artificial Sequence
  synthetic
cttccagtcggggatgtttaca

SEQ ID NO: 16 - DNA, 22 bp, Artificial Sequence
  synthetic
tatttagtgtgataatgccgtt

SEQ ID NO: 17 - DNA, 21 bp, Artificial Sequence
  synthetic
gccaatatttctgtgctgcta

SEQ ID NO: 18 - DNA, 22 bp, Artificial Sequence
  synthetic
ctagtcgtcctaaacatttcac

SEQ ID NO: 19 - DNA, 22 bp, Artificial Sequence
  synthetic
cagactccggtggaatgaagga

SEQ ID NO: 20 - DNA, 22 bp, Artificial Sequence
  synthetic
ggttagtggaccgtgttacata

SEQ ID NO: 21 - DNA, 22 bp, Artificial Sequence
  synthetic
ctcggggcagctcagtacagga

SEQ ID NO: 22 - DNA, 22 bp, Artificial Sequence
  synthetic
cgccaatatttacgtgctgcta